(19) United States
(12) Patent Application Publication
(10) Pub. No.: US 2026/0366986 A1
(43) Pub. Date: Nov. 19, 2026

(54) MULTIPASS SPECTROSCOPIC ABSORPTION CELL AND GAS SENSOR COMPRISING SUCH

(71) Applicant: Automotive Coalition for Traffic Safety, Inc., Leesburg, VA (US)

(72) Inventors: Jonas Orelund, Delsbo (SE); Jakob Kristofers, Järvsö (SE); Hans Martin, Delsbo (SE)

(21) Appl. No.: 19/139,491

(22) PCT Filed: Dec. 14, 2023
(86) PCT No.: PCT/SE2023/051254

§ 371 Date: Jun. 16, 2025

(30) Foreign Application Priority Data
Dec. 16, 2022 (SE) 2251481-4

(51) Int. Cl. G01N 21/03; G01N 21/3504; G01N 33/497

(52) U.S. Cl. CPC G01N 21/031; G01N 21/3504; G01N 33/4972; G01N 2201/0636; G01N 2201/066

(57) ABSTRACT

The present invention relates to a multipass spectroscopic absorption cell (200) and in particular to cells of White-type operating as non-dispersive near infrared gas sensors. The invention further relates to gas sensors (300) and alcohol sensors. The component reflector unit (245; 445) of the multipass spectroscopic absorption cell (200) comprises a first detector (250) and a second detector (260) and a light source (210) arranged to emit a first portion of the light to first reflect in a first concave mirror (220; 420) and forming a first light path ending in the first detector (250), and to emit a second portion of the light to first reflect in the second concave mirror (230; 430) forming a second light path ending in the second detector (260).

### TECHNICAL FIELD

The present invention relates to multipass spectroscopic absorption cells operating as non-dispersive near infrared gas sensors. The invention further relates to alcohol sensors and sensor systems.

### BACKGROUND

Multipass spectroscopic absorption cells are commonly used in various types of gas sensors, not at least as non-dispersive near infrared gas sensors (NDIR sensors). The term “multipass” refers to letting electromagnetic irradiation reflect, preferably multiple times within measuring cell in order to increase the optical path and thereby the sensitivity of a sensor, or measuring system, which the cell is a part of. A particularly useful implementation of a multipass cell is the so called White-cell, proposed by John U White as early as 1942 (Journal of the Optical Society of America, 1942) and used since then. A prior art White-cell is schematically illustrated in FIG. 1a. The prior art White-cell structure 100 is configured by three concave mirrors having the same radius of curvature, a light source 110, and a detector 150. On the opposite surface of the light source 110, first and second reflectors 120 and 130 are positioned in parallel and a third reflector 140 is positioned to face the first and second reflectors 120 and 130. The light source 110 irradiates infrared light 115 at a predetermined angle at any point of the side of the third reflector 140. Thereafter, the infrared light 115 is reflected by the first reflector 120, repetitively reflected between the third reflector 140 and the first and second reflectors 120 and 130, and then incident to the detector 150. The gas to be analyzed is maintained in a cavity and as the infrared light 150 passes through the gas, an output voltage of the detector 150 gives a measure of the concentration of a specific substance present in the gas. The separation of the center of curvature of the first and second reflectors 120 and 130, the angle between an incident optical axis and a longitudinal plane determines the reflection number of incident light and hence the effective optical path length of the device. The White-cell according to FIG. 1 provides eight reflections. However, White cells giving substantially higher numbers of reflections are known in the art. One way of increasing the number or reflections known in the art is to let the light source 110 be arranged with an offset in the transverse direction from the longitudinal optical plane of a measuring cell, providing for a 3D White cell geometry giving at two rows, the upper row 146 and the lower row 147 of light spots on the component reflector unit 140 and 16 reflections as illustrated in FIG. 1b.

One limitation with gas sensors based on White-cell and used in NDIR application is that they are suitable for one wavelength or a rather limited wavelength range corresponding only to a limited number of substances that are possible to detect with one set-up This poses a problem in certain application such as in breath analyzing for detecting and determining a breath concentration of illegal or harmful substances, for example alcohol with the aid of a tracer substance, for example carbon dioxide, since simultaneous detection will not be possible with the same mirrors/detector combination.

U.S. Pat. No. 9,823,237 discloses an integrated breath alcohol sensor system providing simultaneous measurement of alcohol concentration and tracer substance concentration, typically CO₂. A measuring chamber according to U.S. Pat. No. 9,823,237 comprises two detector/mirror set ups, one adapted for alcohol determination and one for CO₂. The optical axis of the two set ups are perpendicular to each other and arranged with their radius of curvature centred in the same plane. Drawbacks includes a complex manufacturing process and the duplication of the mirror parts which increases the production cost.

WO0181901 discloses a White cell of complex geometry compared with the original White cell. The disclosed cell is a 2d cell and may be equipped with a plurality of detectors relating to different ray paths in the cell. Drawbacks includes a very complex geometry with crucial parts spread out in the cell which make the cell difficult and costly to stabilized mechanically and thermally.

### SUMMARY

The object of the present invention is to overcome the drawbacks associated with prior art locking devices. This is achieved by the multipass spectroscopic absorption cell, the gas sensor and the alcoholmeter as defined by the independent claims.

According to one aspect of the invention, a multipass spectroscopic absorption cell is provided. The multipass spectroscopic absorption cell comprises:
a measurement cavity provided with at least one gas inlet and at least one gas outlet and wherein in the measurement cavity comprises:
a main reflector unit comprising a first concave mirror and a second concave mirror;
a component reflector unit comprising a third mirror 240 facing the first concave mirror and the second concave mirror, and wherein
   the first concave mirror and the second concave mirror are spherical mirrors and have the same concave radius of curvature and arranged with their radius of curvature centers aligned in a same longitudinal optical plane, A;
a light source provided adjacent to or in the component reflector unit and arranged to directed emitted light towards the main reflector unit, the light source is in the transverse direction positioned with an offset to the longitudinal optical plane A;
a first detector provided adjacent to or in the component reflector unit,
a second detector provided adjacent to or in the component reflector unit; and in that the light source is arranged to emit a first portion of the light to first reflect in the first concave mirror and forming a first light path ending in the first detector, and to emit a second portion of the light to first reflect in the second concave mirror forming a second light path ending in the second detector.

The distance between the main reflector unit and the component reflector unit may be 95% 105% of the radius of curvature of the first mirror and the second mirror. If the input beam is focused in the plane of the component reflector unit, then each round trip, i.e., after reflection in the main reflector unit, will also be focused in the plane of the component reflector unit.

The third mirror may also be a concave mirror but may also comprise a number of facets, which are arranged in an overall concave configuration, wherein each facet is a flat mirror.

Such a configuration of the third mirror gives a good result when the distance between the main reflector unit and the component reflector unit is approximately the same as the radius of curvature of the first mirror and the second mirror, and might give acceptable results also for a slightly different distance between the main reflector unit and the component reflector unit.

Depending on the configuration of the absorption cell and the number of reflections in the third mirror, the area of the third mirror might be so small that it can be configured as a single flat mirror.

The third mirror may be a spherical mirror and have the same radius of curvature as the first mirror and the second mirror. The first mirror, the second mirror and the third mirror may be arranged with their radius of curvature centers aligned in a same longitudinal optical plane, A. The main reflector unit and the component reflector unit may be arranged at a distance from each other that is essentially the same as the radius of curvature of the first mirror, the second mirror and the third mirror. Such a multipass spectroscopic absorption cell is usually referred to as a White cell.

According to one embodiment of the invention the first detector is provided in a first detector opening in the third mirror and/or the second detector is provided in a second detector opening in the third mirror. By providing the first detector and/or the second detector in openings in the third mirror, a more rigid attachment of the detectors and the light sources in relation to each other and in relation to the third mirror is enabled.

Preferably, the first detector is provided in the component reflector unit, and the second detector is provided in the component reflector unit.

Preferably, the first detector is fixed in relation to the third mirror. Preferably, the second detector is fixed in relation to the third mirror. The fixation of the first and second detector in relation to the third mirror may be achieved in many different ways. The first detector and the second detector may be attached directly to the third mirror. The first detector and the second detector may be fixed to an intermediate element, such as a printed circuit board, wherein the intermediate element is attached to the third mirror. The fixation to an intermediate element may by in addition to attachment of the first detector and the second detector directly to the third mirror. By having the detectors fixed in relation to the third mirror, a steady imaging of the light source on the detectors is achieved. In this way, the amount of light on the detectors will not vary due to vibrations and similar. This should be increasingly important when the number of reflections increases, as this would require a longer third mirror and a larger distance between the light source and the detectors.

The light source may be provided in a light source opening in the third concave mirror. The light source may be fixed in relation to the third mirror. The light source may be attached and fixed directly to the third mirror. The light source may be attached and fixed to the third mirror by means of an intermediate element such as a printed circuit board. The attachment and fixation of the light source by means of an intermediate element may be in addition to attachment of the light source directly to the third mirror. By fixation of the light source in relation to the third mirror, vibrational movements of the light source in relation to the third mirror is minimized.

According to one embodiment of the invention the second light path comprises only one reflection in the main reflector unit and the second detector opening positioned at the position in the third mirror corresponding to the emitted light being reflected only once by the second concave mirror.

According to one embodiment of the invention, the light source is provided in a first half and the first detector is provided in a second half of the component reflector unit, and wherein the second detector is provided in the same half of the component reflector unit as the first detector.

According to one embodiment of the invention, the first portion of the light arranged to first reflect in the first concave mirror and following the first light path represents a major portion of the light emitted by the light source.

According to one embodiment of the invention, first light path comprises a predetermined plurality of reflections between the main reflector unit and the component reflector unit. The first light path may be arranged to be longer than the second light path, preferably at least two times as long and even more preferably at least 4 times as long.

According to one embodiment of the invention, the major portion of the light beam arranged to reflect in the first mirror represents at least 75% of the emitted light, preferably at least 85% of the emitted light, and even more preferably at least 95% of the emitted light.

The desired percentage of the light beam that is to be reflected in the first mirror may be achieved by collimating, directing and/or blocking of light from the light source. The blocking of light may be achieved by shielding of the light source such that the desired percentage of the light is incident on the first mirror. When the light source is arranged in a light source opening in the third concave mirror the shielding may be provided by the light source opening. Mirrors and/or lenses may be used to direct the light to achieve the desired percentage of light in the first light path. The arrangement of the light source and the detectors in openings in the third mirror facilitates the necessary alignment to achieve the desired percentages of light in the first light path and the second light path.

According to one embodiment of the invention the first detector is arranged to measure the light intensity in a first wavelength range and the second detector is arranged to measure the light intensity in a second wavelength range, the second wavelength range being separate from the first wavelength range.

According to one embodiment of the invention the first concave mirror and the second concave mirror are provided as a continuous structure comprised by the main reflector unit, forming a continues mirror surface with two separate curvatures.

According to one embodiment of the invention, the multipass spectroscopic absorption cell comprises:
a main reflector unit comprising a first concave mirror and a second concave mirror arranged so that a main reflector unit back plane, B, is a common symmetrical back plane to the first concave mirror and the second concave mirror;
a component reflector unit comprising a third mirror facing the first concave mirror and the second concave mirror, the component reflector unit having a back plan, C, symmetrical to the third mirror, wherein
the first concave mirror and the second concave mirror are spherical mirrors and have the same concave radius of curvature and arranged with their radius of curvature centers aligned in a same longitudinal optical plane A; and wherein
the component reflector unit back plan, C, is arranged to be transverse to the common longitudinal optical plane, A, and the main reflector unit back plane B and the component reflector unit back plan C are arranged relative each other so that a normal to the main reflector unit back plane B forms an angle, α, to a normal of the component reflector unit back plan C in the longitudinal optical plane, A.

The third mirror may be configured as described above.

The third mirror may be a concave mirror. The first concave mirror, the second concave mirror, and the third concave mirror may be spherical mirrors and have the same concave radius of curvature and be arranged with their radius of curvature centers aligned in a same longitudinal optical plane A.

According to one embodiment of the invention the multipass spectroscopic absorption cell is arranged to provide an upper row of light spots and the lower row of light spots on the component reflector unit and the angle α is selected to cause a shift of the light spots in the lower row so that at least the majority of the light spots in the lower row will be on the same longitudinal position as the light spots of the upper row. The terms, “upper” and lower”, are used merely with reference to the geometry of the embodiments of the invention shown in the drawings and/or during normal operation or mounting of the device/devices and are not intended to limit the invention in any manner.

According to one aspect of the invention, a gas sensor is provided comprising the multipass spectroscopic absorption cell described above. The first wavelength range may relate to a first substance and the second wavelength range substance relate to a second substance.

According to one embodiment of the invention relating to the gas sensor the first detector is arranged to provide measures that relates to the concentrations of the first and second substance wherein the signal relating to the concentration of the first substance which is provided by the first detector relates to a target substance and the signal relating to the concentration of the second substance which is provided by the second detector relates to a tracer substance, and wherein the concentration of the target substance is under normal measuring condition expected to be substantially lower than the concentration of the tracer substance.

According to one aspect of the invention, an alcoholmeter is provided comprising the gas sensor described above, wherein the first substance is alcohol and the second substance is carbon dioxide or water.

Thanks to the invention a compact multipass spectroscopic absorption cell and hence gas sensor and alcoholmeter may be provided that is arranged to simultaneously measure in two different wavelength ranges and thereby detecting two different substances.

One advantage afforded by the present invention is that design is robust and relatively simple to manufacture.

One advantage afforded by embodiments of the present invention is that a second detector may be incorporated in the component reflector unit without substantially increasing the dimensions of the component reflector unit.

Many additional benefits and advantages of the present invention will be readily understood by the skilled person in view of the detailed description below and accompanying drawings.

### DRAWINGS

The invention will now be described in more detail with reference to the appended drawings, wherein

FIGS. 1a-b schematically illustrate a prior art multipass spectroscopic absorption cell of White-type;

FIGS. 2a-g schematically illustrate the multipass spectroscopic absorption cell according to the invention;

FIG. 3 schematically illustrates a gas sensor device according to the invention; and

FIGS. 4a-g schematically illustrate one embodiment of the multipass spectroscopic absorption cell according to the invention;

All the figures are schematic, not necessarily to scale, and generally only show parts, which are necessary in order to elucidate the respective embodiments, whereas other parts may be omitted or merely suggested. Any reference number appearing in multiple drawings refers to the same object or feature throughout the drawings, unless otherwise indicated.

### DETAILED DESCRIPTION

Terms such as “top”, “bottom”, upper“, lower”, “below”, “above” etc are used merely with reference to the geometry of the embodiment of the invention shown in the drawings and/or during normal operation or mounting of the device/devices and are not intended to limit the invention in any manner.

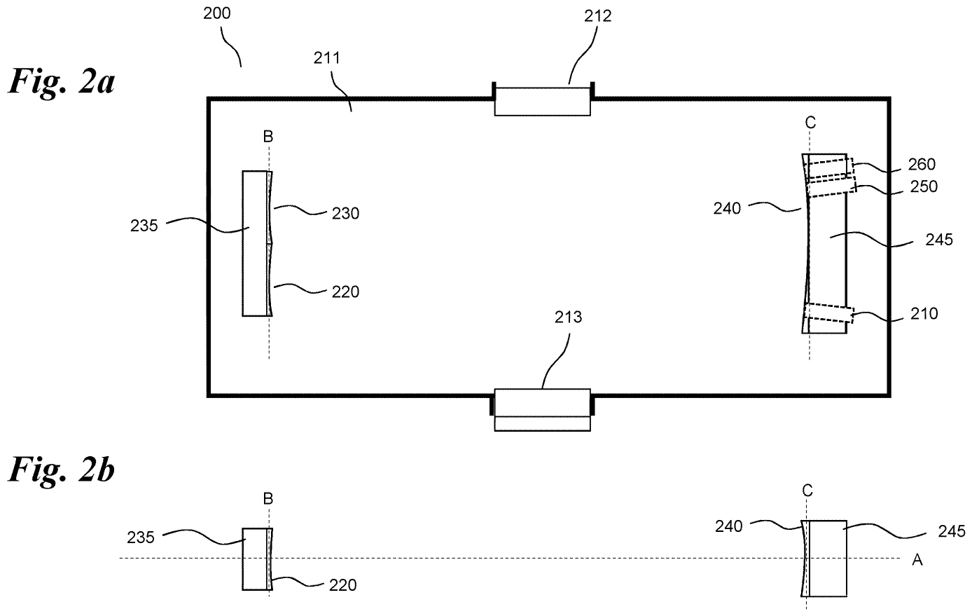

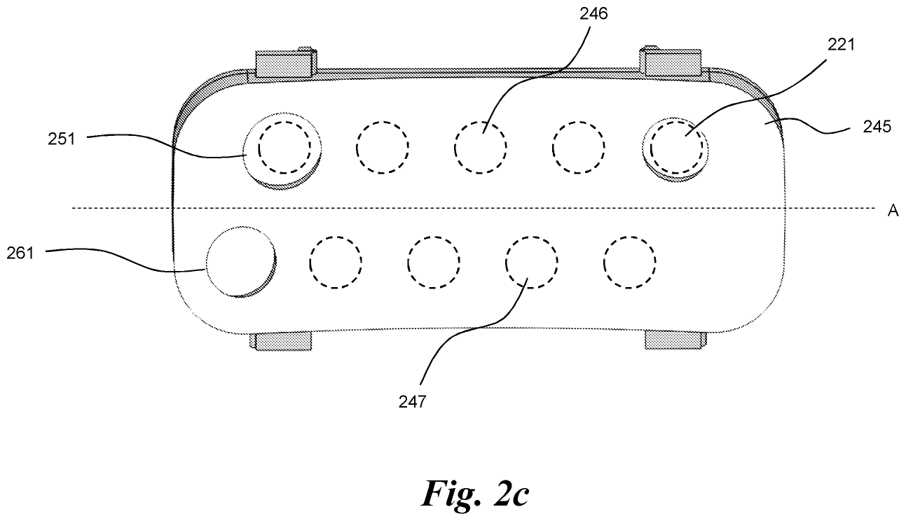

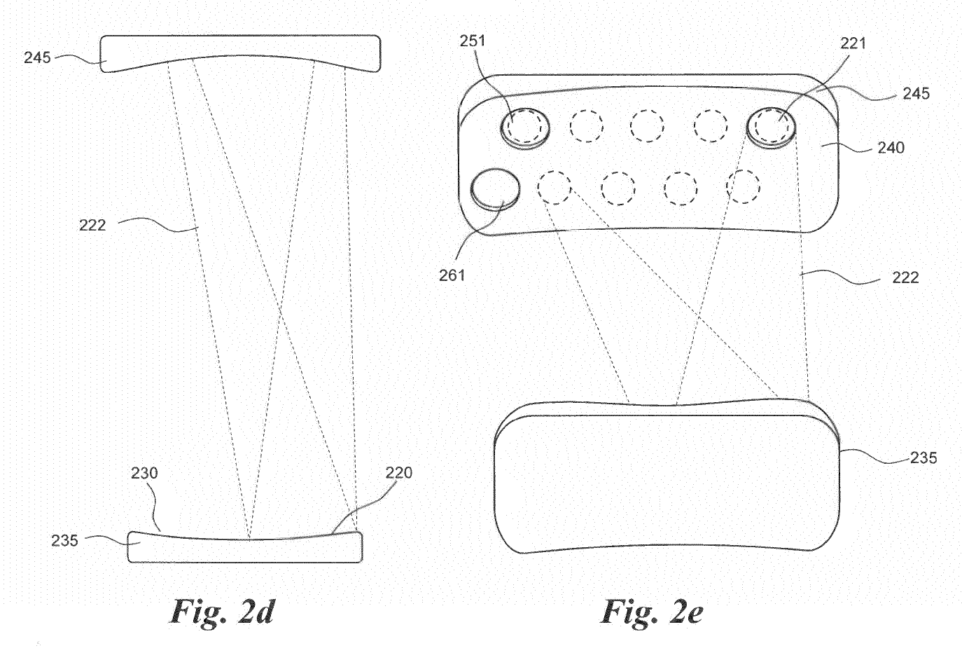

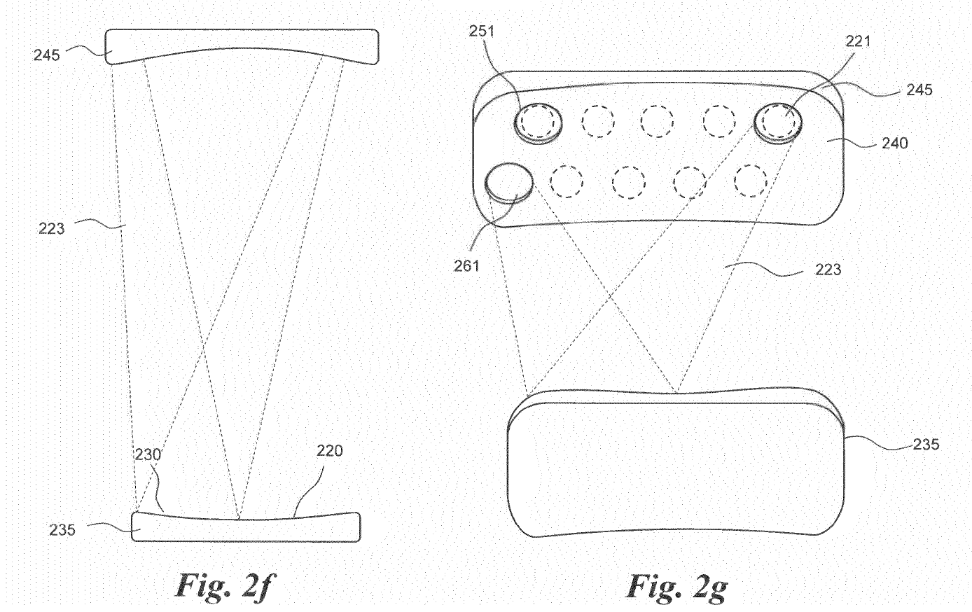

The multipass spectroscopic absorption cell 200 according to the present invention is schematically depicted in FIG. 2a-h. As depicted in FIG. 2a in a top view the multipass spectroscopic absorption cell 200 comprises a measurement cavity 211 provided with at least one gas inlet 212 and at least one gas outlet 213. Arranged in the measurement cavity 211 is a main reflector unit 235 comprising a first concave mirror 220 and a second concave mirror 230, and a component reflector unit 245 comprising a third concave mirror 240 facing the first concave mirror 220 and the second concave mirror 230. The first concave mirror 220, the second concave mirror 230 and the third concave mirror 240 are spherical mirrors and have the same concave radius of curvature. The radius of curvature is typically in the order of 2-15 cm, determining the minimum physical length of the system, as well as the achievable optical path-length The first concave mirror 220, the second concave mirror 230 and the third concave mirror 240 are arranged with their radius of curvature centres aligned in a same longitudinal optical plane, A as schematically illustrated in FIG. 2b in a sideview and in FIG. 2c in an elevated view of the component reflector unit 245. The second concave mirror 230 and the second concave mirror 230 are arranged essentially adjacent to each other and in parallel in the main reflector unit 235. This may be seen as the first concave mirror 220 and the second concave mirror 230 having a common back plane transverse to the longitudinal optical plane, the main reflector unit back plane, B, through the periphery of the spheres associated with the first concave mirror 220 and the second concave mirror 230, respectively. Similarly, a component reflector unit back plan also transverse to the longitudinal optical plane, C, may be defined including the periphery of the sphere associated with the third concave mirror 240 and being symmetrical with regards to the third concave mirror 240. The main reflector unit back plane B and the component reflector unit back plan C are perpendicular to the longitudinal optical plane A and the main reflector unit back plane B is parallel to the component reflector unit back plan C.

The distance between the main reflector unit back plane B and the component reflector unit back plan C is according to the White cell geometry close to the radii of the curvature of the first concave mirror 220, the second concave mirror 230 and the third concave mirror 240.

Alternatively, the third mirror may comprise a number of facets, which are arranged in an overall concave configuration, wherein each facet is a flat mirror. Such a configuration of the third mirror gives a good result when the distance between the main reflector unit and the component reflector unit is approximately the same as the radius of curvature of the first mirror and the second mirror. In the following, the third mirror will be referred to as a concave mirror.

Both the component reflector unit 245 and the main reflector unit 235 are typically of a rectangular shape extending further in the longitudinal direction than in the transverse direction. The optimal minimum shape of the active area of the main reflector unit 235 depends on the actual active emitter area, which by a light collector ideally will be magnified on to the main reflector unit 235. Typically, this image will be 10 to 40 times larger than the emitter source. The third concave mirror 240 may have the dimensions 10×35 mm and the first concave mirror 220 and the second concave mirror 230 about 10×15 mm each.

The first concave mirror 220 provided adjacent to the second concave mirror 230 should be understood that the first concave mirror 220 and first concave mirror 220 may abut. However, they may also be arranged with a small distance in-between. According to one embodiment the first concave mirror 220 and the first concave mirror 220 are provided as a continuous structure comprised by the main reflector unit 235, essentially forming a mirror unit with two separate curvatures. Such a monolithic dual mirror design may be advantageous since it will firmly fix the relative positions of the two radii of curvature centers, which is a very sensitive parameter for the propagation of the rays in the multi-pass cell. Any small change, d, in this parameter value will on the beam exit move the final output position by d*N (N=number of single passes in the cell), with a consequent transmission signal loss.

A light source 210 is arranged adjacent to or in the component reflector unit 245 and is directed so that the emitted light is essentially directed towards main reflector unit 235. The light source 210 is in the transverse direction positioned with an offset to the longitudinal optical plane A, providing for a 3D White cell geometry giving at least two rows, the upper row 246 and the lower row 247 of light spots on the component reflector unit 245 as illustrated in FIG. 2c.

According to one embodiment of the invention, the light source 210 is arranged with its light emitting part coinciding with the imaginary sphere associated with the third concave mirror 240. According to one embodiment, the light source 210 is provided outside of the third concave mirror 240 although preferably integrated in the component reflector unit 245. According to one embodiment, the light source 210 is provided in a light source opening 221 in the third concave mirror 240. Typically, and preferably, the light source 210 is provided closer to one edge in the elongated direction than to the center of the third concave mirror 240.

In the multipass spectroscopic absorption cell 200 according to the invention the component reflector unit 245 is provided with a first detector 250 and a second detector 260 adapted for measuring the intensity of incoming light. According to one embodiment at least one of the first detector 250 and the second detector 260 are provided in an opening in the third concave mirror 240. According to one embodiment, the first detector 250 is provided in a first detector opening 251 and the second detector 260 is provided in a second detector opening 261. Preferably, the first detector 250 and the second detector 260 are aligned with the imaginary sphere associated with the third concave mirror 240.

In the multipass spectroscopic absorption cell 200 according to the invention the light source 210 is arranged to emit a first portion of the light to first reflect in the first concave mirror 220 and to emit a second portion of the light to first reflect in the second concave mirror 230 as schematically illustrated in FIG. 2e-f in a top view (e) and an elevated view (f). The light that first reflects in the first concave mirror 220 is arranged to form a first light path 222 comprising a plurality of reflections between the main reflector unit 235 and the component reflector unit 245 and which ends in the first detector 250. In the figure, for simplicity, only a first reflection is illustrated. The plurality of reflections typically and preferably is a predetermined number of reflections, such as 16 reflections giving an optical length in the order of 1 m. The light that first reflects in the second concave mirror 230 is arranged to form a second light path 223, which ends in the second detector 260 as schematically illustrated in FIG. 2g-h in a top view (g) and an elevated view (h).

According to one embodiment the second light path 223 comprises only one reflection in the main reflector unit 235 and the second detector opening 261 and second detector 260 is positioned at the position in the third concave mirror 240 corresponding to the emitted light being reflected only once by the second concave mirror 230.

According to one embodiment, the second light path 223 comprises a predetermined number of reflections between the main reflector unit 235 and the component reflector unit 245. Accordingly, the second detector opening 261 and the second detector 260 are positioned at a position relating to the predetermined number of reflections.

The curvature of the concave mirrors has been described as spherical but might have a slight ellipsoidal form in order to correct for anastigmatic defects. Such corrections are well known in the art and the term spherical mirrors used here within encompasses such variations and corrections.

According to one embodiment, schematically illustrated in FIG. 2c, the light source 210 is provided in a first half 246 and the first detector 250 is provided in a second half 247 of the component reflector unit 245. The second detector 260 is provided in the same half of the component reflector unit 245 as the light source 210.

According to one embodiment, the first portion of the light arranged to first reflect in the first concave mirror 220 and following the first light path 222 represents a major portion of the light emitted by the light source 210. Consequently, the second portion of the arranged to first reflect in the second concave mirror 230 and light following the second light path 222 represents a minor portion of the light emitted by the light source 210. The relation between the major and the minor light portion should be chosen with regards to the requested S/N requirement for the two detectors The major portion of the emitted light, i.e. the light in the first light path, may for example represents at least 75% of the emitted light, for example at least 85% of the emitted light, or for example at least 95% of the emitted light. The desired percentage of emitted light in the first light path may be controlled by positioning of the light source 210 in relation to the light source opening 221 and the size and shape of the light source opening 221.

According to one embodiment, the first detector 250 is arranged to measure the light intensity in a first wavelength range and the second detector 260 is arranged to measure the light intensity in a second wavelength range, the second wavelength range being separate from the first wavelength range. The first wavelength range may relate to a first substance and the second wavelength range may relate to a second substance. The first detector 250 may be arranged to provide a signal relating to a target substance and the second detector 260 arranged to provide a signal relating to a tracer substance. The term “target substances” refers to the substance for which the concentration is of primary interest. The term “tracer substance” refers to a substance which concentration is interesting primarily to facilitate the measurement of the concentration of the target substance, for example to remove influences of a disturbing factor or relate the measurement to specific conditions. Typically, the concentration of the target substance is expected to be substantially lower than the concentration of the tracer substance. In a case that the multipass spectroscopic absorption cell 200 is utilized in a breath alcohol sensor system the first substance/target substance is alcohol and the second substance/tracer substance is carbon dioxide or water.

According to one embodiment, the first detector 250 and the second detector 260 are arranged to measure the light intensity in the same wavelength range and thereby arranged to provide a measure of the concentration of the same substance. In such a case the arrangement with two separate light paths and two detectors may be utilized to expand the sensitivity range of the multipass spectroscopic absorption cell 200 in that the first light path 222 (the longer light path) provides accurate measurements for a lower concentration range and second light path (the shorter light path) provides accurate measurements for a higher concentration range of the measured substance. Alternatively, or in combination, a setup with the first detector 250 and the second detector 260 arranged to measure the light intensity in the same wavelength range is primarily utilized to provide redundancy and/or to facilitate function control or calibration.

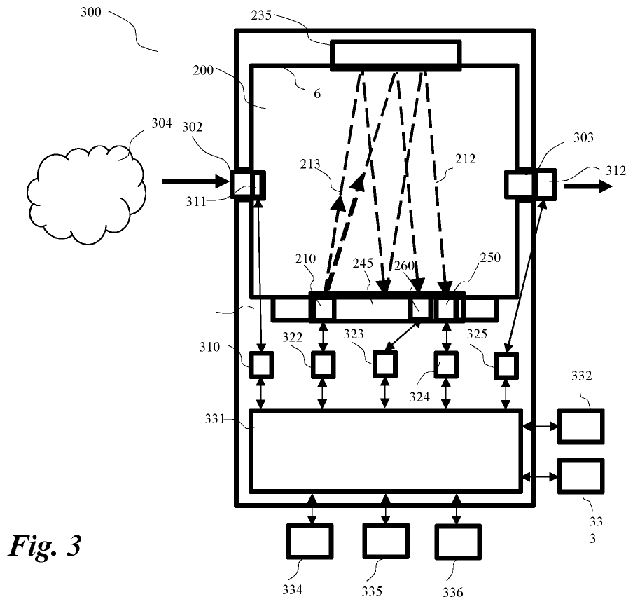

The multipass spectroscopic absorption cell 200 may be incorporated in a large variety of gas sensor devices. A gas sensor device 300 according to the invention is schematically illustrated in FIG. 3. The gas sensor device 300 is suitable to be used as, but not limited to, a breath analysis device, in particular to measure the alcohol content in a breath sample with the aid of a tracer substance such as water vapor or carbon dioxide. Below a breath analysis device will be described as a non-limiting example. Other gas sensor devices wherein the multipass spectroscopic absorption cell 200 is the central part includes, but is not limited to devices arranged for measurements wherein there is an overlap between the target substance and water vapor absoptions. Example of such devices are gas sensors for measuring nitrous oxide, methane and carbon dioxide.

The gas sensor device 300 comprises the multipass spectroscopic absorption cell 200 with the measurement cavity 211 the gas inlet 212 and gas outlet 213 comprised in a housing 301. The gas inlet 212 and gas outlet 213 of the measurement cavity 211 are in connection with inlet 302 and outlet 303 of the housing 301. A gas sample 304, for example a breath sample, is during use drawn into measurement cavity 211 for analyzing its content of tracer and other substances, for example intoxicating substances. The inlet 302 may include a heater 311 arranged to heat the gas sample. Heating the gas sample may be of importance due to the temperature dependence of the measurements and also to avoid condensation on parts of the measurement cavity 211. A fan 312 is typically provided to provide an even air flow through the measurement cavity 211. The fan 312 may be provided at the outlet 304 of the housing 301.

The sensor signals according to one embodiment of the invention are generated by non-dispersive infrared (NDIR) spectroscopy, in which case the light source 210 is an IR source and the first and second detectors 250, are IR-sensitive detectors. Suitable light sources and detectors are commercially available. According to one embodiment the first detector 250 is tuned to the absorption spectra of the intoxicating substance, typically ethyl alcohol, and the second detector 260 is tuned to the absorption spectra of CO₂ or water. CO₂ has a strong absorption peak at a wavelength of 4.26 μm, whereas H₂O has relatively broad peaks at 2.5-2.8 and 5.3-7.6 μm. Ethyl alcohol has a specific peak at 9.5 μm not shared by any of the most frequent interfering substances, but with a small cross sensitivity to CO₂.

The IR source, light source 210 and the first and second detector 250, 260 are preferably operating synchronously, using a repetition and sampling rate exceeding the frequency bandwidth required for the analysis of breath signals. Synchronous operation using phase-locking techniques is preferable from the point of view of noise and interference suppression. A repetition and sampling rate of 5 Hz may be considered as a lower limit which is compatible with the response time of MEMS-based (Micro Electro-Mechanical Systems) IR emitters, and photovoltaic or thermopile IR detectors.

Interface electronic circuitry 321, 322, 323, 324, 325 is controlling the heater 311, light source 210, first detector 250, second detector 260, and the fan 312, respectively. Each of these subsystems include electronic drive and power supply control devices adapting the different functionalities to be manageable by a central processing unit, CPU, 310 which is a general-purpose digital microcontroller. Also included in the gas sensor device 300 are memory devices 332, 333 for permanent and temporary storage of information.

The CPU 310 and the memory devices 332 and 333 are arranged to control the transfer and storage of data including sensor signals during the analysis described below, and to control the method steps and in real time execute the mathematical operations described below. As appreciated by the skilled person other configurations are possible to provide the controlling and computational functions here indicated and the above described should be regarded as an illustrative example and one embodiment. One alternative embodiment is that the breath analysis system is integrated with other measurement and/or controlling systems in a vehicle and the functions of the CPU 331 is provided by a main CPU in the vehicle also handling other tasks and the detectors and the other units of the gas sensor device 300 communicates via a vehicle bus system or similar.

The gas sensor device 300 may comprise or be connected to a human/machine interface (HMI) unit 334 for audiovisual communication between the system and a user. The HMI unit 334 typically comprises communication means via microphone/loudspeaker, touch screen or other input/output devices. It has the capability of visual, verbal or symbolic communication of specific requests and classification results to the subject. Alternatively, the gas sensor device 300 may be connected to and utilizes an existing infotainment system in the vehicle for the human/machine interface.

In automotive applications the system may include a vehicle drivability control unit 335 directly connected to the vehicle control system, providing the “alcolock” functionality. In vehicles driven by fossil fuel, the unit 335 may control the ignition, and in other types of vehicles it will control other basic driving mechanisms.

A communication unit 336 for wireless information exchange between the gas sensor device 300 and other external units, preferably over the internet, may be provided and is useful in a wide range of applications.

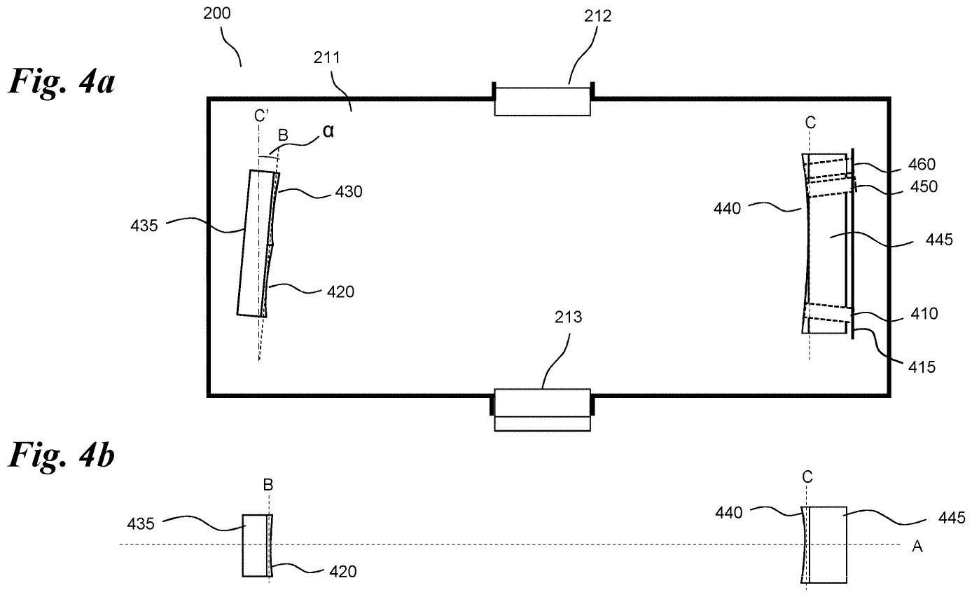

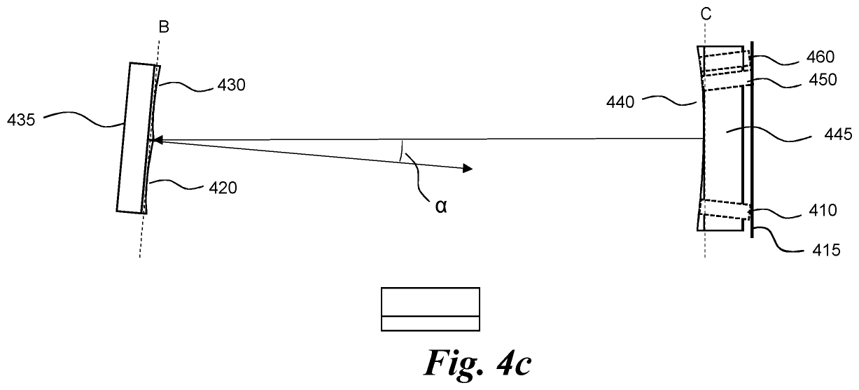

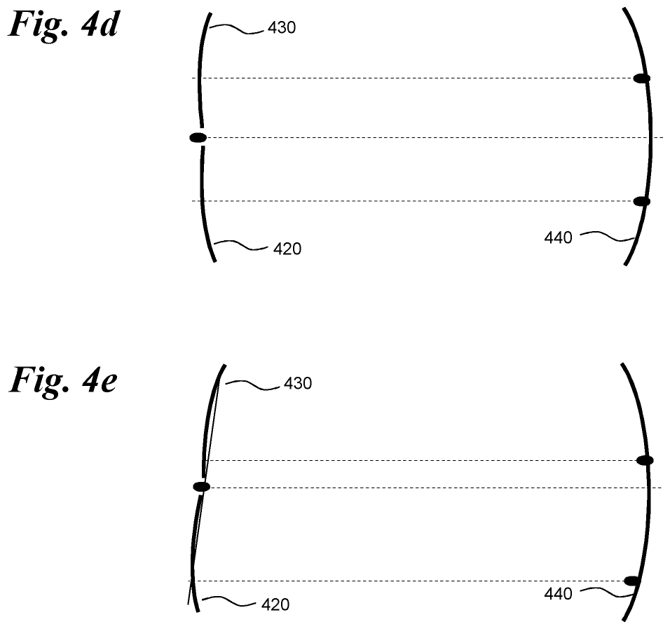

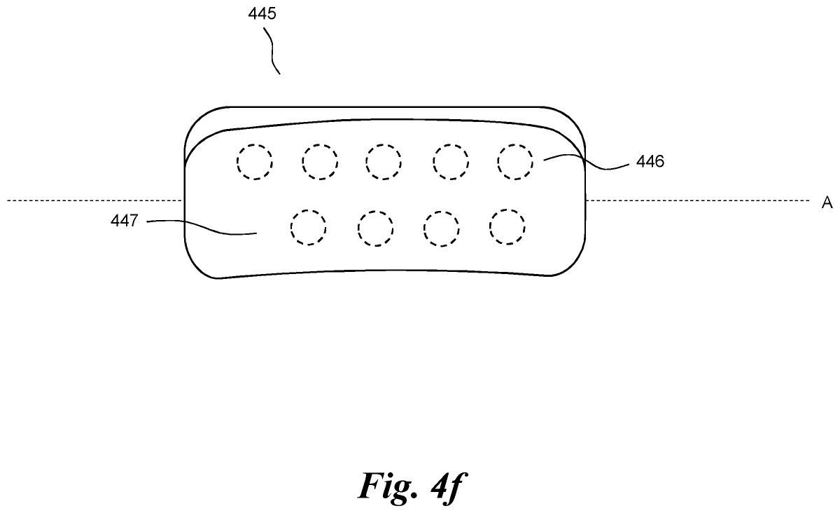

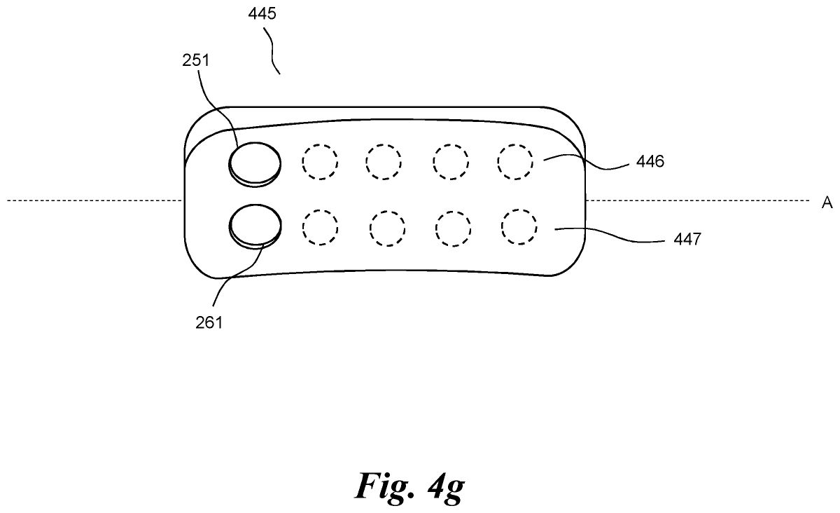

In one embodiment of the invention, the above described dual wavelength setup is provided without significantly increasing the size of the White-cell, in particular without increasing the size of the mirrors. One realization of a multipass spectroscopic absorption cell 200 of White-type according to such embodiment is schematically depicted in FIG. 4a-g. As depicted in FIG. 4a in a top view the multipass spectroscopic absorption cell 200 comprises a measurement cavity 211 provided with at least one gas inlet 212 and at least one gas outlet 213. Arranged in the measurement cavity 211 is a main reflector unit 435 comprising a first concave mirror 420 and a second concave mirror 430, and a component reflector unit 445 comprising a third concave mirror 440 facing the first concave mirror 420 and the second concave mirror 430. The first concave mirror 420, the second concave mirror 430 and the third concave mirror 440 are spherical mirrors and have the same concave radius of curvature. The radius of curvature is typically in the order of 2-15 cm, determining the minimum physical length of the system, as well as the achievable optical path-length. The first concave mirror 420, the second concave mirror 430 and the third concave mirror 440 are arranged with their radius of curvature centres aligned in a common longitudinal optical plane, A as schematically illustrated in FIG. 4b in a sideview and in FIG. 4c in an elevated view of the component reflector unit 445 and in FIG. 4d elevated view of the main reflector unit 435. The second concave mirror 430 and the second concave mirror 430 are arranged essentially adjacent to each other and in parallel in the main reflector unit 435. This may be seen as the first concave mirror 420 and the second concave mirror 430 having a common back plane, the main reflector unit back plane, B, through the periphery of the spheres associated with the first concave mirror 420 and the second concave mirror 430, respectively. Similarly, a component reflector unit back plan, C, may be defined including the periphery of the sphere associated with the third concave mirror 440 and being symmetrical with regards to the third concave mirror 440. The component reflector unit back plan C is arranged to be transverse to the common longitudinal optical plane, A. The main reflector unit back plane B and the component reflector unit back plan C are arranged with an angle, α, relative each other such that a normal to the main reflector unit back plane B forms an angle, α, to a normal of the component reflector unit back plan C in the longitudinal optical plane, A only. This tilting of the main reflector unit 435 with regards to the component reflector unit 445 is illustrated in FIG. 4a with the main reflector unit back plane B forming the angle, α, to the component reflector unit back plan C plane as seen from above, wherein an imaginary plane C′ is indicated close to the plane B. In the side view, FIG. 4b, the projections of the planes B and C are parallel, and both are perpendicular to the longitudinal optical plane A. In FIG. 4c the normals to the main reflector unit back plane B and the component reflector unit back plan C and the angle α are indicated. The effect of the tilted main reflector unit 435 is further illustrated in FIGS. 4d-e illustrating the relations between the optical axis of the first concave mirror 420, the second concave mirror 430 and third concave mirror 440, wherein d) is a prior art mirror arrangement without tilt and b) is the tilted optical arrangement according to the embodiment.

The distance between the main reflector unit back plane B and the component reflector unit back plan C is according to the White cell geometry close to the radii of the curvature of the first concave mirror 420, the second concave mirror 430 and the third concave mirror 440.

Both the component reflector unit 445 and the main reflector unit 435 are typically of a rectangular shape extending further in the longitudinal direction than in the transverse direction. The optimal minimum shape of the active area of the main reflector unit 435 depends on the actual active emitter area, which by a light collector ideally will be magnified on to the main reflector unit 435. Typically, this image will be 10 to 40 times larger than the emitter source. The third concave mirror 440 may have the dimensions 10×35 mm and the first concave mirror 420 and the second concave mirror 430 about 10×15 mm each.

The first concave mirror 420 provided adjacent to the second concave mirror 430 should be understood that the first concave mirror 420 and first concave mirror 420 may abut. However, they may also be arranged with a small distance in-between. According to one embodiment, the first concave mirror 420 and the first concave mirror 420 are provided as a continuous structure comprised by the main reflector unit 435, essentially forming a mirror unit with two separate curvatures. Such a monolithic dual mirror design may be advantageous since it will firmly fix the relative positions of the two radii of curvature centers, which is a very sensitive parameter for the propagation of the rays in the multi-pass cell. Any small change, d, in this parameter value will on the beam exit move the final output position by d*N (N=number of single passes in the cell), with a consequent transmission signal loss.

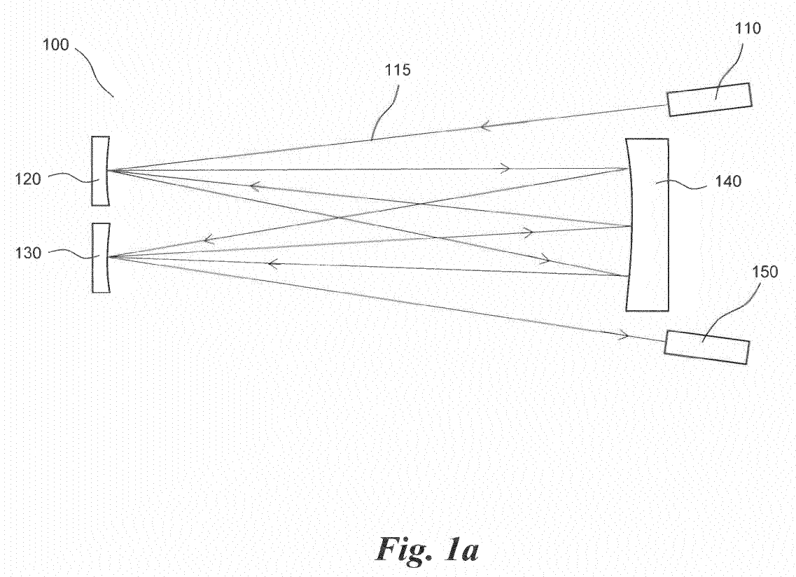

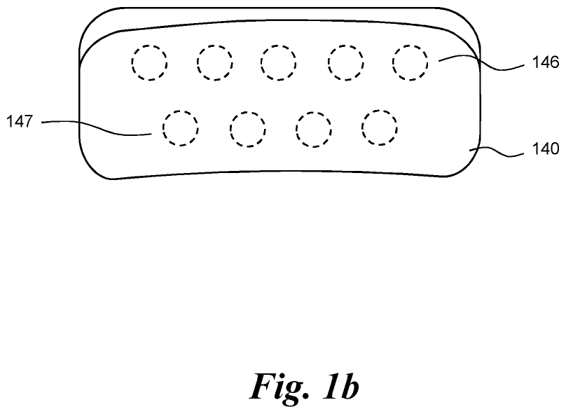

A light source 410 is arranged adjacent to or in the component reflector unit 445 and is directed so that the emitted light is essentially directed towards main reflector unit 435. The light source 410 is in the transverse direction positioned with an offset to the longitudinal optical plane A, providing for a 3D White cell geometry during use giving at least two rows, the upper row 446 and the lower row 447 of light spots on the component reflector unit 445 as illustrated in FIG. 4d. As a consequence of the angle α between the main reflector unit 435 and the component reflector unit 445 according to the present invention the positions in the longitudinal direction between the light spots in the lower row 447 will be shifted, whereas the light spots of the upper row 446 will not be shifted as compared to a comparable White-cell with main reflector unit back plane B being parallel with the component reflector unit back plan C as illustrated in FIG. 1b. The shift of the light spots the lower row 447 will depend, in addition to the angle α, on the dimensions of the mirrors and the number of passes between the mirrors (the optical length) by straightforward trigonometry. The skilled person will, having knowledge of the geometry of the White-Cell and having gain the insight of this description, easily calculate an angle α corresponding to a specific shift. For the multipass spectroscopic absorption cell 200 with the third concave mirror 440 having dimensions 10×35 mm and the first concave mirror 420 and the second concave mirror 430 about 10×15 mm each and with an optical length of 100 mm and 2-5 passes, suitable angles ranges from 2° to 15°.

According to one embodiment of the invention the angle α is selected to cause a shift of position of the light spots in the lower row 447 so the positions in the longitudinal direction will be essentially the same as the light spots in the upper row 446 as schematically illustrated in FIG. 4g. Thereby, the second detector 460 and the corresponding second detector opening 261 may be positioned below the first detector 450 and the corresponding first detector opening 251 and the component reflector unit 445 with two detectors may be provided without any increase, or very limited increase, in size. This corresponds to an angle α of 6° for the cell according to the example described above. As shown in FIGS. 4a and 4c, the light source 410, the first detector 450 and the second detector 460 are arranged on a common printed circuit board 415. The printed circuit board 415 is attached to the component reflector unit 445.

According to one embodiment of the invention, the light source 410 is arranged with its light emitting part coinciding with the imaginary sphere associated with the third concave mirror 440. According to one embodiment, the light source 410 is provided outside of the third concave mirror 440 although preferably integrated in the component reflector unit 445.

According to one embodiment, the light source 410 is provided in a light source opening 221 in the third concave mirror 440. Typically, and preferably, the light source 410 is provided closer to one edge in the elongated direction than to the center of the third concave mirror 440.

The curvature of the concave mirrors has been described as spherical but might have a slight ellipsoidal form in order to correct for anastigmatic defects. Such corrections are well known in the art and the term spherical mirrors used here within encompasses such variations and corrections.

The embodiment described with reference to FIGS. 4a-g is readily implemented in the gas sensor device 300 described with reference to FIG. 3.

The embodiments described above are to be understood as illustrative examples of the system and method of the present invention. It will be understood that those skilled in the art that various modifications, combinations and changes may be made to the embodiments. In particular, different part solutions in the different embodiments can be combined in other configurations, where technically possible.

1. A multipass spectroscopic absorption cell (200) comprising a measurement cavity (211) provided with at least one gas inlet (212) and at least one gas outlet (213) and wherein in the measurement cavity (211) comprises:
a main reflector unit (235; 435) comprising a first concave mirror (220; 420) and a second concave mirror (230; 430);
a component reflector unit (245; 445) comprising a third mirror 240 facing the first concave mirror (220; 420) and the second concave mirror (230; 430), and wherein the first concave mirror (220; 420), and the second concave mirror (230; 430) are spherical mirrors and have the same concave radius of curvature and arranged with their radius of curvature centers aligned in a same longitudinal optical plane, A;
a light source (210) provided adjacent to or in the component reflector unit (245; 445) and arranged to direct emitted light towards the main reflector unit (235; 435), the light source (210) is in the transverse direction positioned with an offset to the longitudinal optical plane A; and
a first detector (250) provided adjacent to or in the component reflector unit (245; 445), the multipass spectroscopic absorption cell characterized by
a second detector (260) provided adjacent to or in the component reflector unit (245; 445); and in that the light source (210) is arranged to emit a first portion of the light to first reflect in the first concave mirror (220; 420) and forming a first light path (222) ending in the first detector (250), and to emit a second portion of the light to first reflect in the second concave mirror (230; 430) forming a second light path (223) ending in the second detector (260).

2. The multipass spectroscopic absorption cell (200) according to claim 1, wherein the first detector (250) is provided in a first detector opening (251) in the third mirror (240; 440) and/or the second detector (260) is provided in a second detector opening (261) in the third mirror (240; 440).

3. The multipass spectroscopic absorption cell (200) according to claim 1, wherein the second light path (223) comprises only one reflection in the main reflector unit (235; 435) and the second detector opening (261) positioned at the position in the third mirror (240; 440) corresponding to the emitted light being reflected only once by the second concave mirror (230; 430).

4. The multipass spectroscopic absorption cell (200) according to claim 1, wherein the light source (210) is provided in a first half (246) and the first detector (250) is provided in a second half (247) of the component reflector unit (245; 445), and wherein the second detector (260) is provided in the same half of the component reflector unit (245; 445) as the first detector (250).

5. The multipass spectroscopic absorption cell (200) according to claim 1, wherein the first portion of the light arranged to first reflect in the first concave mirror (220; 420) and following the first light path (212) represents a major portion of the light emitted by the light source (210).

6. The multipass spectroscopic absorption cell (200) according to claim 1, wherein the first light path (212) comprises a predetermined plurality of reflections between the main reflector unit (235; 435) and the component reflector unit (245; 445).

7. The multipass spectroscopic absorption cell (200) according to claim 6, wherein the first light path (212) is longer than the second light path (223), preferably at least two times as long and even more preferably at least 4 times as long.

8. The multipass spectroscopic absorption cell (200) according to claim 5, wherein the major portion of the light beam (212) arranged to reflect in the first mirror (220; 420) represents at least 75% of the emitted light, preferably at least 85% of the emitted light, and even more preferably at least 95% of the emitted light.

9. The multipass spectroscopic absorption cell (200) according to claim 1, wherein the first detector (250) is arranged to measure the light intensity in a first wavelength range and the second detector (260) is arranged to measure the light intensity in a second wavelength range, the second wavelength range being separate from the first wavelength range.

10. The multipass spectroscopic absorption cell (200) according to claim 1, wherein the first concave mirror (220; 420) and the second concave mirror (230; 430) are provided as a continuous structure comprised by the main reflector unit (235; 435), forming a continues mirror surface with two separate curvatures.

11. The multipass spectroscopic absorption cell (200) according to claim 1, comprising:
wherein the first concave mirror (420) and the second concave mirror (430) are arranged so that a main reflector unit back plane, B, is a common symmetrical back plane to the first concave mirror (420) and the second concave mirror (430);
wherein the third mirror (440) is concave and wherein the component reflector unit has a back plan, C, symmetrical to the third concave mirror (440), wherein
the first concave mirror (420), the second concave mirror (430) and the third concave mirror (440) are spherical mirrors and have the same concave radius of curvature and arranged with their radius of curvature centers aligned in a same longitudinal optical plane A; wherein
the component reflector unit back plan, C, is arranged to be transverse to the common longitudinal optical plane, A, and the main reflector unit back plane B and the component reflector unit back plan, C, are arranged relative each other so that a normal to the main reflector unit back plane, B, forms an angle, a, to a normal of the component reflector unit back plan, C, in the longitudinal optical plane, A

12. The multipass spectroscopic absorption cell (200) according to claim 11, wherein the multipass spectroscopic absorption cell (200) is arranged to provide an upper row of light spots (446) and the lower row (447) of light spots on the component reflector unit (445) and the angle a is selected to cause a shift of the light spots in the lower row (447) so that at least the majority of the light spots in the lower row will be on the same longitudinal position as the light spots (446) of the upper row.

13. A gas sensor (300) comprising the multipass spectroscopic absorption cell (200) according to claim 1.

14. The gas sensor (300) according to claim 13, wherein the first wavelength range relates to a first substance and the second wavelength range substance relates to a second substance.

15. The gas sensor (300) according to claim 14, wherein the first detector (250) is arranged to provide measures that relates to the concentrations of the first and second substance wherein the signal relating to the concentration of the first substance which is provided by the first detector (250) relates to a target substance and the signal relating to the concentration of the second substance which is provided by the second detector (260) relates to a tracer substance, and wherein the concentration of the target substance is under normal measuring condition expected to be substantially lower than the concentration of the tracer substance.

16. An alcoholmeter comprising the gas sensor (300) according to claim 13, wherein the first substance is alcohol and the second substance is carbon dioxide or water.
